(19) United States
(12) Patent Application Publication
(10) Pub. No.: US 2006/0287533 A1
(43) Pub. Date: Dec. 21, 2006

(54) SOLVENT CONTAINING 1,2,5-THIADIAZOLE COMPOUND, AND METHOD FOR EXTRACTING AN ORGANIC COMPOUND USING THE SOLVENT

(75) Inventor: Kuniaki Tatsuta, Tokyo (JP)

Correspondence Address: AKERMAN SENTERFITT, P.O. BOX 3188, WEST PALM BEACH, FL, 33402-3188, US

(73) Assignee: Waseda University, Tokyo (JP)

(21) Appl. No.: 11/463,279

(22) Filed: Aug. 8, 2006

(30) Foreign Application Priority Data
Jan. 28, 2004 (JP) 2004-050416

(51) Int. Cl. C07D 285/08

(52) U.S. Cl. 548/128

(57) ABSTRACT

The object of the present invention is to provide a solvent which is free from the risk of destroying the ozone layer surrounding the Earth, hardly toxic, and free from the risk of causing environment pollution problems by polluting underground water or air.
The solvent contains at least one chosen from 1,2,5-thiadiazole compounds represented by formula (1) below: 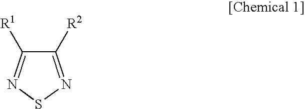
(where R¹ and R² each represent hydrogen or methyl group).

### BACKGROUND OF THE INVENTION

1. Field of the Invention

The present invention relates to a solvent containing a 1,2,5-thiadiazole compound. The present invention relates more specifically to a solvent containing a 1,2,5-thiadiazole compound which is suitably used as a solvent for extracting a desired compound from a mixture comprising various compounds, a solvent serving as a medium in various chemical reactions, a solvent for washing the parts of electronic/mechanical devices, a solvent for separating a resin or the like, or a diluent for diluting paint/ink, and to a method for extracting an organic compound using a solvent of the present invention.

2. Description of the Related Art

To extract a desired compound from a mixture comprising various compounds, to serve as a medium in various chemical reactions, to wash the parts of electronic/mechanical devices, to separate a resin or the like, or to dilute paint/ink, halogenated hydrocarbons such as dichloromethane, chloroform, trichloroethylene, dichloroethane, or the like have been widely used.

Halogenated hydrocarbons, because of their being highly capable of dissolving organic compounds and chemically stable, have been used as a versatile solvent for the extraction of various organic compounds in diverse applications. The halogenated hydrocarbon, because of its having a specific gravity larger than 1 and being immiscible to water, is particularly preferable when it is used as a solvent for extracting a desired compound from an aqueous solution, because then the organic phase constitutes an underlying layer which will enable the ready extraction of the desired compound, and improve the work efficiency.

In addition, halogenated hydrocarbons, because of their being highly capable of dissolving organic compounds, inert to reagents, and highly selective to reactions, have been widely used as a solvent in acid/base based reactions, halogen involving reactions, or metal catalyst based reactions. Furthermore, the halogenated hydrocarbon, because of its having a specific gravity larger than 1 and being immiscible to water, will facilitate the separation of reaction products and recovery of the solvent after the completion of reaction, which will lead to the improved work efficiency.

Still further, halogenated hydrocarbons, because of their being highly capable of dissolving oils/fats and waxes, and chemically stable, have been widely used as a cleaning solvent for degreasing the parts of electronic/mechanical devices, washing print-boards, and washing glass substrates.

Still further, halogenated hydrocarbons, because of their being highly capable of dissolving resins such as organic adhesives, resists or the like, have been widely used as a separating solvent.

Still further, halogenated hydrocarbons, because of their being highly capable of dissolving organic materials such as paints, inks, resins, or colorants, and dispersing them, and chemically stable, have been widely used as a diluent for diluting organic materials (see, for example, Organic Solvent Association (ed.), “Pocketbook for Solvents,” Revised Edition, Ohm Publishing, Co., 1997, pp. 252-330).

### SUMMARY OF THE INVENTION

Although halogenated hydrocarbons have excellent properties as described above, halogen atoms serving as a constituent of those halogenated hydrocarbons pose a serious problem, because when they are released in the air, they will act as a catalyst in the destruction of ozone layers surrounding the Earth. Moreover, halogenated hydrocarbons are not only harmful themselves, but when they are discharged into the environment, they may spoil the environment by polluting underground water/air. Thus, there is a strong demand for an alternative solvent compensating for the disadvantages inherent to the halogenated hydrocarbons.

To solve the above problem, the present invention aims to provide a solvent containing no halogen atom, and being highly capable of dissolving organic compounds and chemically stable, having a specific gravity larger than 1, immiscible to water and capable of acting as an extraction solvent, reaction medium, cleaning solvent, separating solvent, or diluent without exerting adverse effects on the ozone layer, being essentially free from the risk of giving toxic effects to organisms and causing environmental problems. Another object of the present invention is to provide a method for extracting an organic compound using the above extraction solvent.

The present inventors found that a 1,2,5-thiadiazole compound is highly capable of dissolving a variety of organic compounds, is excellent in chemical stability, has a specific gravity larger than 1, is immiscible to water, and does not contain any halogen atom, and that, since the compound is thus free from the risk of destroying the ozone layer, or hardly causes environmental problems by polluting underground water or air, it will serve as an excellent extraction solvent, reaction medium, cleaning solvent, separating solvent, or diluent.

According to a first aspect of the present invention, there is provided a solvent containing at least one chosen from 1,2,5-thiadiazole compounds represented by formula (1) below: 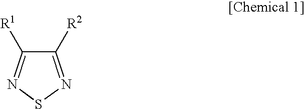
(where R¹ and R² each represent hydrogen or methyl group). According to the present invention, the 1,2,5-thiadiazole compound represented by formula (1) above is preferably 1,2,5-thiadiazole represented by formula (2) below:

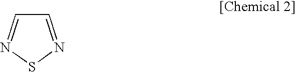

The solvent of the present invention preferably serves as a solvent for extraction, medium for reaction, solvent for cleaning, solvent for separation, or solvent for dilution.

According to a second aspect of the present invention, there is provided a method for extracting an organic compound using a solvent of the present invention.

The extraction method of the present invention is preferably a method for extracting an organic compound using a solvent comprising contacting a solid or liquid mixture comprising the organic compound with an extraction solvent as described in any one of claims 1 to 3, thereby extracting the organic compound, and separating the organic compound from the extraction solvent containing that organic compound.

Alternatively, the extraction method of the present invention is preferably a method for extracting an organic compound using a solvent wherein the mixture is an aqueous solution containing the organic compound. The organic compound is preferably an organic compound having a polar group as its molecular element.

According to the present invention, there is provided a solvent which does not contain any halogen atom, being thus free from the risk of destroying the ozone layer, and hardly causing environmental problems by polluting underground water or air, is highly capable of dissolving organic compounds, and excellent in chemical stability, has a specific gravity larger than 1, and is immiscible to water, particularly an extraction solvent, reaction medium, cleaning solvent, separating solvent, or diluent having properties as described above.

### DESCRIPTION OF THE PREFERRED EMBODIMENTS

The present invention will be described in detail below. A first feature of the present invention is a solvent containing at least a 1,2,5-thiadiazole compound represented by formula (1). In the formula, R¹ and R² each represent hydrogen or methyl group.

A 1,2,5-thiadiazole compound as represented by formula (1) is highly capable of dissolving a variety of organic compounds, is chemically stable, is immiscible to water, has a boiling point equal to or lower than 150° C., and has a specific gravity larger than 1. Although the compound has a specific gravity larger than 1, it does not contain any halogen atom, and thus is free from the risk of destroying the ozone layer, and hardly causes toxicity problems and environment pollution problems.

Among 1,2,5-thiadiazole compounds, 1,2,5-thiadiazole (boiling point: 94° C.; specific gravity: 1.26) is preferred because of its ready availability, dissolving ability, and adequate boiling point. The 1,2,5-thiadiazole compound as represented by formula (1) is a publicly known compound, and may be made, for example, by a method as described in U.S. Pat. No. 3,440,246 (Tetrahedron Lett., 1966, P.

1263), etc.

The solvent of the present invention contains at least 1,2,5-thiadiazole. The content of a 1,2,5-thiadiazole compound in the solvent of the present invention is usually 10 wt % or higher with respect to the total weight of the solvent, preferably 50 wt % or higher.

The solvent of the present invention may contain, in addition to a 1,2,5-thiadiazole compound, one or two or more liquid organic compound(s).

Preferred additional liquid organic compounds include, for example, aliphatic hydrocarbons such as n-pentane, 2-methylbutane, 2,2-dimethylpropane, n-hexane, 2-methylpentane, 3-methylpentane, 2,2-dimethylbutane, 2,3-dimethylbutane, n-heptane, 2-methylhexane, 3-methylhexane, 2,3-dimethylpentane, 2,4-dimethylpentane, n-octane, 2,2,3-trimethylpentane, 2,2,4-trimethylpentane, n-nonane, n-decane, etc.; alicyclic hydrocarbons such as cyclopentane, methylcyclopentane, cyclohexane, methylcyclohexane, ethylcyclohexane, cyclooctane, etc.; aromatic hydrocarbons such as benzene, toluene, xylene, etc.; alcohols such as methanol, ethanol, n-propanol, isopropanol, n-butanol, methyl cellosolve, carbitol, etc.; ethers such as diethyl ether, diisopropyl ether, dibutyl ether, tetrahydrofuran, monoglyme, diglyme, 1,2-dimethoxyethane, dioxane, etc.; ketones such as acetone, methylethyl ketone, 2-pentanone, 3-pentanone, cyclopentanone, cyclohexanone, etc.; esters such as ester formate, ester acetate, etc.; nitoriles such as acetonitrolie, etc.; amids such as N,N-dimethylformamide, N,N-dimethylacetoamide, hexamethylphosphoric triamide, N-methylpyrrolidone, etc.; organic nitrogen compounds such as nitrobenzene, etc.; organic sulfur compounds such as dimethyl sulfoxide, sulforan, etc.; organic silicon compounds such as tetramethyl silane, tetraethyl silane, methoxytrimethyl silane, ethoxytrimethyl silane, hexamethyl disiloxane, octamethyl cyclotetrasiloxane, etc.; cyclic hydrocarbons such as bicyclo[2,2,1]heptane, bicyclo[2,2,2]octane, pinane, dihydrocyclopentadiene, tetrahydrodicyclopentadiene, etc.; terpene-based hydrocarbons such as limonene α-pinene, β-pinene, dipentene, etc., but are not limited to those compounds.

A solvent of the present invention may further include, in addition to a 1,2,5-thiadiazole compound and liquid organic compound described above, a still other component as appropriate. The additional component may be determined as appropriate depending on whether the solvent is used for extraction, as a reaction medium, for washing, for separation, or for dilution.

The 1,2,5-thiadiazole compound represented by formula (1) or (2) above is highly capable of dissolving a wide variety of chemical substances, is chemically stable in the presence of various reactive substances, is free from the risk of destroying the ozone layer, is hardly toxic, and hardly causes environmental problems via pollution. Accordingly, the solvent of the present invention is useful for extracting a desired organic compound from a solid or liquid mixture comprising the organic compound.

Since the compound in the present invention has a specific gravity larger than 1, and is immiscible to water, when it is used for extracting a desired matter from an aqueous solution, the organic phase containing the desired matter forms an underlying layer. Therefore, it is readily possible to separate the desired matter from the underlying layer during the extraction step which will improve the efficiency of the extraction step.

The content of a 1,2,5-thiadiazole compound in the extraction solvent of the present invention is usually 50 wt % or higher, preferably 70 wt % or higher, more preferably 90 wt % or higher.

Suitable organic compounds to be extracted by the solvent of the present invention are not limited to any specific compounds, as long as they are soluble to a 1,2,5-thidiazole compound. However, from the viewpoint of using the extraction solvent of the present invention in place of conventional halogenated hydrocarbon solvents, suitable organic compounds to be extracted by the solvent of the present invention may include organic compounds highly soluble to halogenated hydrocarbons such as chloroform, dichloromethane, and the like.

Suitable organic compounds to be extracted by the solvent of the present invention may include organic compounds which include a polar group(s) in their molecular structure. The polar group used herein refers to oxygen atom, nitrogen atom, sulfur atom, etc., that has a different electronegativity from that of carbon atom. Suitable polar groups may include, for example, amide group, carboxyl group, ester group, hydroxyl group, carbonyl group, amino group, nitro group, cyano group, alkoxy group, mercapto group, alkylthio group, etc.

The organic compound having a polar group(s) in its molecular structure may include, for example, active ingredients contained in naturally occurring substances, medicines and agrochemicals, industrial chemicals, flavoring agents, etc., and intermediates obtained during the production of those substances, chemicals and agents that have one or two or more polar groups in their molecular structure. The molecular weight of those organic compounds is not limited to any specific range, but is usually in the range of 100 to 1000, preferably 100 to 500. If an organic compound to be extracted by a solvent of the present invention has a streogenic carbon atom in its molecular structure, the organic compound may be a mixture of optical isomers, or consist of either one of the optical isomers. Even if such an organic compound consisting of one optical isomer is used for extraction, racemization will not occur during extraction.

The 1,2,5-thiadiazole compound represented by formula (1) or (2) above is highly capable of dissolving a wide variety of chemical substances, is chemically stable in the presence of various reactive substances over a wide temperature range, is free from the risk of destroying the ozone layer, is hardly toxic, and hardly causes environmental problems via pollution. Accordingly, the solvent of the present invention is suitably used as a reaction medium in various reactions.

The content of a 1,2,5-thiadiazole compound in the medium for reaction in the present invention is usually 30 wt % or higher, preferably 50 wt % or higher.

When the solvent of the present invention is used as a medium for reaction, suitable additional liquid organic compounds described above may include, for example, aliphatic hydrocarbons, aromatic hydrocarbons, alicyclic hydrocarbons, ethers, nitriles, and amides.

Suitable reactions for which a solvent of the present invention can serve as a medium are not limited to any specific reactions, but preferably include reactions involving the use of a halogenated hydrocarbon such as dichloromethane or the like, from the viewpoint of using the solvent of the present invention in place of a conventional halogenated hydrocarbon solvent.

Such suitable reactions may include, for example, various polymerization reactions such as anionic polymerization, cationic polymerization, radical polymerization, etc., oxidation reaction, reducing reaction, nucleophilic substitution reaction, electronphilic substitution reaction, translocation reaction, addition reaction, elimination reaction, addition/elimination reaction, insertion reaction, isomerization reaction, decomposition reaction, solvent-added decomposition reaction, coupling reaction, metathesis reaction, condensation reaction, stereogenic element synthesis reaction, pericyclic reaction, photochemical reaction, electrochemical reaction, radical reaction, ring-opening reaction, ring-closing reaction, cleavage reaction, hydrogenation reaction, esterification reaction, halogenation reaction, carbonylation reaction, heterocycle synthesis reaction, dehydration reaction, hydration reaction, metal catalyst-mediated reaction, rare earth metal-mediated reaction, etc.

The 1,2,5-thiadiazole compound represented by formula (1) or (2) above is highly capable of dissolving a wide variety of chemical substances, such as machine oil, abrasion oil, lubricating oil, rust-proof oil, rosin, waxes, higher fatty acid, resist, and other organic compounds, is free from the risk of destroying the ozone layer, is hardly toxic, and hardly causes environmental problems via pollution. Accordingly, the solvent of the present invention is suitably used for washing oil/fat, resin, paint, lacquer, varnish, etc.

The content of a 1,2,5-thiadiazole compound in the washing solvent of the present invention is usually 10 wt % or higher, preferably 50 wt % or higher, or more preferably 70 wt % or higher.

When the solvent of the present invention is used as a washing solvent, suitable additional compounds described above may include anionic surfactants, cationic surfactants, amphoteric surfactants, non-ionic surfactants, rust-proof agents, abrasion agents, etc. The addition amount of the additional component is usually in the range of 0 to 30 wt % with respect to the total weight of the washing solvent.

The washing solvent of the present invention is suitably used for washing every kind of products including fibrous products made of metal, ceramic, glass, plastic, or elastomer produced in such fields as precision machine industry, automobile industry, aircraft industry, heavy machine industry, metal machining industry, metal assembling industry, iron/steel works industry, non-iron industry, steel pipe industry, heat treatment industry, metal plating industry, metallurgy industry, optical machine industry, business machine industry, electronics industry, electrics industry, plastics industry, glass industry, ceramics industry, printing industry, fiber industry, cleaning business, etc.

Suitable articles to be washed by the washing solvent of the present invention may include a wide variety of articles produced in a variety of fields of industries, for example, the parts of various electronics products such as printing boards, metal masks, lead frames, magnetic heads, ceramic capacitors, parts of hard disk drive, parts of video disk player, wafers, semiconductor chips, bi-metals, thermo-modules, piezoelectric elements, hybrid IC's, parts of liquid crystal display, pin-and-ball grid alleys, etc.; the parts of electric appliances such as parts of electrode, relay parts, press parts, micro-motors, heat-exchangers, fine tubes, resistors, parts of broadcasting equipment, parts of lighting equipment, parts of electric products, electronic guns of cathode ray oscilloscope, parts of antenna, parts of signal handling devices, cables of made of hoop material, etc.; the parts of precision machines such as parts of camera, lens cover of replacement photoelectric element, parts of watch, lenses, parts of watch frame, parts of copier, polygon mirror, etc.; the parts of automobile body such as springs, parts of brake, parts of key, parts of engine, nozzle for fuel ejection, gears, shafts, pairing, sintered metal parts, etc.; the parts of various machines such as parts of pump, parts of compressor, reaction apparatus, parts of valve, etc.; rolled plates (metal); pipes (copper); chromatography columns; thin tubes and joints of tubes (metal); photosensitive drums, etc.

Soils which can be eliminated by the washing solvent of the present invention is not limited to any specific soils. However, removable soils way include, for example, oils such as abrasion oil, machine oil, lubricating oil, rust-proof oil, annealing oil, heat treatment oil, rolling oil, extension oil, forging oil, machining oil, processing oil, press-processing oil, punching oil, cast removing oil, extraction oil, assembling oil, lining oil, synthetic oils (silicon-, glycol- and ester-based oils), etc.; greases; waxes; paint, ink, rubber, varnish, coating materials, abrasives, adhesives, thinners of adhesives, superficial layer separating agents, oils/fats, mold releaser to separate mold from die, asphalt pitch, hand grease, finger print, proteins, flux after welding, resist, light reflection prevention membrane of resist, protective membrane of optical lens, organic photosensitive agent, photosensitive resin (photosensitive resist), masking agent, compounds, surfactants, solder paste, cut scraps, saw dust, lens pitch (lens abrasive), metal powder, metal abrasive, lubricant, various resins (melamine resin, polyurethane, polyester, epoxy resin, rosin resin), processing scraps, burr, resin powder, inorganic powder, paper powder, puff powder, particles, ionic soil, dust, etc.

The washing solvent of the present invention may be used successfully in combination with various washing methods such as solvent immersion, exposure to ultrasonic waves, shaking, solvent spraying, solvent showering, exposure to solvent steam, towel wiping, etc. During washing, additional physical procedures such as stirring, vibration, brushing, etc. may be employed as needed.

The 1,2,5-thiadiazole compound represented by formula (1) or (2) above is highly capable of dissolving organic adhesives, resins serving as resists, organic insulating materials, and paint/ink, is free from the risk of destroying the ozone layer, is hardly toxic, and hardly causes environmental problems via pollution. Accordingly, the solvent of the present invention is suitably used as an agent for separating such an organic material as described above.

An illustrative example of such a separating solvent may include an etchant used in photoetching: a photoresist is applied on a silicon wafer, the wafer is exposed to light, and the etchant is used to remove unnecessary (e.g., unexposed) portions of the photoresist.

Suitable photoresists to be etched by the solvent of the present invention are not limited to any specific ones, and may be arbitrarily chosen from conventional positive or negative type light exposure resists, infra-UV light exposure resists, X-ray or electron beam exposure resists, etc. Known main ingredients of the materials of those resists may include a variety of resins such as novolak resins, cyclic rubbers, polycinnamate resins, (meta-)acrylic resins, (meta-)acrylate copolymers, polyhydroxystylene resins, etc. The etchant of the present invention is effective for any one chosen from the regists described above. To enhance the etching effect of the etchant of the present invention, an etching enhancing agent selected from benzenesulfonic acid, toluenesulfonic acid, xylenesulfonic acid, phenolsulfonic acid, and alkylbenzenesulfonic acid such as methyl-, propyl-, heptyl-, octyl-, decyl-, and dodecyl-benzenesulfonic acid may be used in combination with the etchant. The addition amount of etching enhancing agent is usually in the range of 5 to 30 wt % with respect to the total weight of etchant. To enhance the etching effect of the etchant of the present invention, a surfactant such as a non-ionic surfactant, anionic surfactant or cationic surfactant may be used in combination as needed.

Another illustrative example of the application of a separating solvent of the present invention may relate to a case where a semiconductor material, crystal material, electronics part material, magnetic material, or piezoelectric element material is bonded via an adhesive to a processing jig, and the material is processed, that is, cut, abraded, or polished, and involve using the separating solvent for separating the material from the jig.

It is readily possible to separate the material from the jig by immersing the assembly comprising the material bonded via adhesive to the jig in the solvent of the present invention, keeping it there for a sufficient period of time, and extracting the material from the solvent.

Suitable semiconductor materials to be separated by the solvent of the present invention may include crystal materials such as silicon, gallium arsenide, gallium phosphide, etc. Suitable electronics part related materials may include crystal materials, quartz crystal, quartz, glass, etc. Suitable magnetic materials may include ferrite, samarium, cobalt, etc. Suitable magnetic and piezoelectric materials may include magnetic head, etc.

Suitable organic adhesives may include, for example, synthetic resin-based adhesives such as epoxy resin-based adhesives, polyurethane-based adhesives, etc.; elastomer-based adhesives such as chloroprene-based adhesives, nitrile rubber-based adhesives, stylene butadiene rubber-based adhesives, thermoplastic elastomer-based adhesives, etc.; and mixture adhesives such as mixture adhesive of vinyl resin and phenol resin, mixture adhesive of nitrile rubber and phenol resin, mixture adhesive of chloroprene rubber and phenol resin, mixture adhesive of epoxy resin and phenol resin, mixture adhesive of epoxy resin and soluble nylon, etc.

The 1,2,5-thiadiazole compound represented by formula (1) or (2) above is highly capable of dissolving and dispersing various organic materials such as paints, ink, lubricants, mold lubricant, water-repellent and oil-repellent agent, oil, grease, resin, pigments, etc., is chemically stable, has an appropriate boiling point, is free from the risk of destroying the ozone layer, is hardly toxic, and hardly causes environmental problems via pollution. Accordingly, the solvent of the present invention is suitably used as a diluent for diluting such an organic material as described above.

The solvent of the present invention is also suitably used, for example, as a solvent for dissolving a coating material used for forming a thin film on an information recording medium or organic photosensitive material, solvent for crystallization or recrystallization, solvent for recycling used rubber, plasticizer, lubricant, wetting agent, solvent for gel permeation chromatography (GPC), solvent for high performance liquid chromatography (HPLC), solvent for column chromatography, dissolving agent of polymers, solvent for casting, solvent for spin coating, etc.

A second aspect of the present invention relates to a method for extracting an organic compound using a solvent of the present invention. The extracting method using a solvent of the present invention comprises, for example, liquid-liquid extraction where an extraction solvent is added to a solution containing an organic compound, the extraction solvent being immiscible to the solvent of the solution, such that the organic compound is extracted by the added solvent based on the difference in partition coefficient between the two solvents, or solid-liquid extraction where an extraction solvent is added to a solid mixture containing an organic compound, such that the organic compound is extracted by the added solvent. Since the solvent of the present invention is immiscible to water and easily separable from water, it is particularly useful when used as a solvent for extracting an organic compound from an aqueous solution of the organic compound.

Since the solvent of the present invention has a specific gravity equal to or larger than 1, when it is used as an extraction solvent for extracting a desired compound, e.g., 1,2,5-thidiazole compound from its aqueous solution, the phase (organic phase) containing the 1,2,5-thidiazole compound forms an underlying layer. Therefore, it is readily possible to extract the underlying layer from beneath the system, which will improve the efficiency of the extraction operation.

Illustrative examples of liquid-liquid extraction may include (i) method comprising adding an extraction solvent of the present invention to an aqueous solution containing an organic compound, the organic compound being soluble to water and the extraction solvent with, however, its solubility to the extraction solvent being higher than its solubility to water, stirring the system thoroughly, then resting the system until the system is divided into two phases, i.e., a water phase and an organic phase, and extracting the organic phase; and (ii) method comprising adding acid to an aqueous solution of a salt of acidic organic compound such as carboxylic acid thereby releasing the acidic compound, or base to an aqueous solution of a salt of basic organic compound thereby releasing the basic compound, and extracting the thus released acidic or basic organic compound using an extraction solvent of the present invention. With the latter method, extraction may be repeated several times. The extraction temperature is usually kept in the range of 20-100° C., preferably 0-90° C., more preferably 20-50° C.

When liquid-liquid extraction is performed on a laboratory scale, a separatory funnel may be used. When liquid-liquid extraction is performed on a mass-production scale, a publicly known mixer-settler, multi-stage mixer/decanter type contactor, or gravity-based separation column type contactor may be used.

When a separatory funnel is used, an appropriate amount of a solvent of the present invention is added to an aqueous solution of a compound to be extracted, and the system is shaken thoroughly. The system is then allowed to stand until it is divided into two phases, organic phase and water phase. The organic phase is separately extracted.

The publicly known mixer-settler incorporates a large separatory funnel, and is used essentially in the same manner as is the separatory funnel used in a laboratory. The multi-mixer/decanter type contactor is a non-stirring extractor, and its extraction tower typically expels, from its lower level, a light solution (e.g., aqueous solution) having a smaller specific gravity and, from its upper level, a heavy solution (e.g., solution based on a solvent of the present invention) having a larger specific gravity. According to this system, a lighter solution flows upward while a heavier solution flows downward, and thus two solutions are brought into contact with each other in the tower. Then, solutes dissolved in the light and heavy solutions are redistributed to the light and heavy solutions according to their partition coefficients to the respective solutions. When the tower incorporates a multiple stage of porous plates, the light solution passes through tiny holes of the porous plates to turn into small droplets which go upward, and come into contact with the heavy solution. A multitude of droplets pass through tiny holes of a next porous plate to turn into droplets and go further upward. Repeated formation of droplets and joining with the other solution enable the effective contact of the two solutions. The gravity-based separation column type contactor is an extracting unit based on mechanical agitation, and suitable methods for mechanical agitation may include agitation with a rotating paddle, pulse-transmitted vibration, etc.

An illustrative example of solid-liquid extraction may include a method comprising thoroughly mixing an extraction solvent of the present invention with a solid mixture containing an organic compound to be extracted, extracting the organic compound by the extraction solvent, and removing insoluble impurities from the extract solution, for example, by filtration. Incidentally, it is desirable prior to the extraction operation to pulverize the solid mixture, so as to improve the efficiency of extraction. Furthermore, the extraction solvent may be heated during extraction.

Extraction units suitably used for solid-liquid extraction may include a publicly known extracting unit comprising filer cloth or porous plate at the bottom, where a solid object comprising a compound to be extracted is allowed to rest on the filtering device, and an extraction solvent of the present invention is allowed to circulate through the unit. If solid-liquid extraction should be performed on a mass-production scale, a continuous extracting unit as described in PCT Japanese Translation Patent Publication No. 9-510913 may be suitably used.

In any method, the basic operation consists of separating a layer of an extraction solvent (solution) of the present invention, and drying the extract solution as needed, to remove the extraction solvent of the present invention. The residue thus obtained is washed by solvent, recrystallized, and purified by a known purification method such as column chromatography, distillation, etc., to isolate the target compound. If the extracted substance is a naturally occurring substance thermally unstable, removal of the extraction solvent may be achieved by employing special maneuver such as vaporization under reduced pressure, reduction of the partial pressure of solvent by blowing water vapor into the solution (steam stripping), etc. The used extraction solvent of the present invention may be recovered with a solvent recovering unit, purified as needed, for example, by distillation, and used again as an extraction solvent.

### EXAMPLES

The examples of the present invention will be disclosed below.

### Example 1

For 1,2,5-thiadiazole of the present invention, its compatibility with other solvents was studied. The test consisted of mixing 1,2,5-thiadiazole with another solvent at a volume ratio of 1/1 (2 ml), allowing the system to stand at room temperature, and visually inspecting the compatibility of the former with the latter. The results are shown in Table 1.

Circle: fully compatible to give a uniform mixture

Triangle: giving a white turbidity

X-mark: being divided into two phases

TABLE 1

| Solvent | Compatibility |
|---|---|
| Water | X |
| Methanol | ◯ |
| Ethanol | ◯ |
| Ethyl acetate | ◯ |
| Acetone | ◯ |
| Dimethylformamide | ◯ |
| Acetonitrile | ◯ |
| Benzene | ◯ |
| Xylene | ◯ |
| Toluene | ◯ |
| n-Hexane | ◯ |

### Example 2

A 10 ml of 1,2,5-thiadiazole and 10 ml of water were put into a separatory funnel, and the system was shaken vigorously, to be allowed to stand. The system was clearly divided into two phases, an upper water phase and lower 1,2,5-thiadiazole phase.

### Example 3

The dissolving capability of 1,2,5-thiadiazole of the present invention was studied. A 20 mg of each of compounds 1 to 20 was transferred into a sample vial, to which 1,2,5-thiadiazole was added at room temperature with stirring. The addition was continued until the compound was completely dissolved, and the addition amount of the solvent was determined. The results are shown in Table 2.

### Comparative Example 1

The same procedure as in Example 3 was repeated except that 1,2,5-thiadiazole was replaced with chloroform. The amount of the solvent required for completely dissolving the compound was determined. The results are shown in Table 2.

### Comparative Example 2

The same procedure as in Example 3 was repeated except that 1,2,5-thiadiazole was replaced with diethylether. The amount of the solvent required for completely dissolving the compound was determined. The results are shown in Table 2.

TABLE 2

| | | | | | | Comp. Ex. | |
|---|---|---|---|---|---|---|---|
| | Compound | Example 3 | | Comp. Ex. 1 | | 2 | |
| 1 | 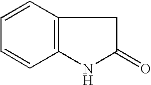 | 100 | μl | 400 | μl | 2000 | μl |
| 2 | 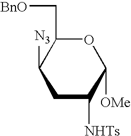 | 140 | μl | 240 | μl | 1600 | μl |
| 3 | 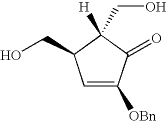 | 160 | μl | 200 | μl | Insoluble | |
| 4 | 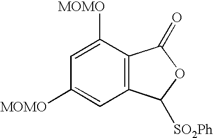 | 1000 | μl | Hardly soluble | | Hardly soluble | |
| 5 | 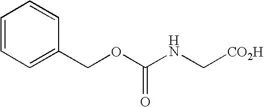 | 1000 | μl | 2000 | μl | 2000 | μl |
| 6 | 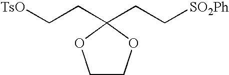 | 1000 | μl | 2000 | μl | Hardly soluble | |
| 7 | 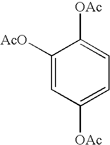 | 100 | μl | 200 | μl | 2000 | μl |
| 8 | 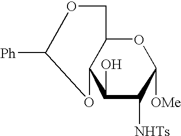 | 200 | μl | 240 | μl | 1600 | μl |
| 9 | 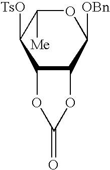 | 20 | μl | 200 | μl | 6000 | μl |
| 10 | 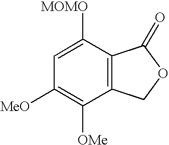 | 200 | μl | 400 | μl | Hardly soluble | |
| 11 | 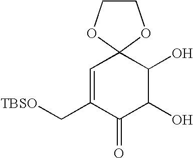 | 200 | μl | 400 | μl | Hardly soluble | |
| 12 | 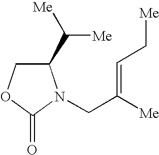 | 200 | μl | 200 | μl | 200 | μl |
| 13 | 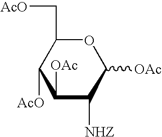 | 100 | μl | 100 | μl | 2000 | μl |
| 14 | 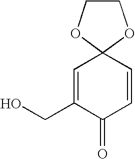 | 100 | μl | 100 | μl | 2000 | μl |
| 15 | 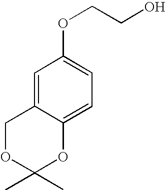 | 100 | μl | 100 | μl | 2400 | μl |
| 16 | 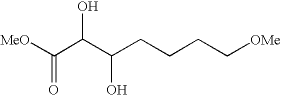 | 100 | μl | 100 | μl | 200 | μl |
| 17 | 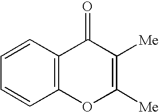 | 40 | μl | 40 | μl | 4000 | μl |
| 18 | 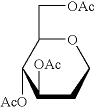 | 200 | μl | 200 | μl | 200 | μl |
| 19 | 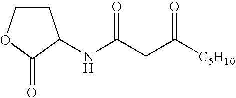 | 100 | μl | 100 | μl | 4800 | μl |

| | | | | | | |
|---|---|---|---|---|---|---|
| 20 | 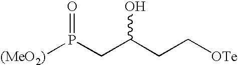 | 200 | μl | 200 | μl | Hardly soluble |

### Example 4

The extracting activity of 1,2,5-thiadiazole of the present invention was studied. A 2 mg of each of compounds 1 to 20 was dissolved in 100 μl of 1,2,5-thiadiazole, to which 100 μl of distilled water was added, and the system was subjected to separation operation. The ratio of the content of the compound in water phase against the counterpart in organic phase was determined using TLC, and the results are shown in Table 3.

### Comparative Example 3

The same procedure as in Example 4 was repeated except that 1,2,5-thiadiazole was replaced with chloroform, to determine the comparative extracting activity of chloroform. The results are shown in Table 3.

### Comparative Example 4

The same procedure as in Example 4 was repeated except that 1,2,5-thiadiazole was replaced with diethylether, to determine the comparative extracting activity of diethylether. The results are shown in Table 3.

TABLE 3

| | | Example | Comp. Ex. | Comp. Ex. |
|---|---|---|---|---|
| | Compound | 4 | 3 | 4 |
| 1 | 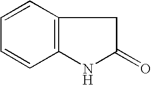 | 1/100 | 1/10 | 1/5 |
| 2 | 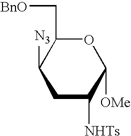 | 0/100 | 1/50 | 1/4 |
| 3 | 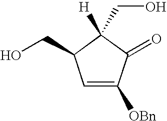 | 1/5 | 4/5 | Hardly soluble |
| 4 | 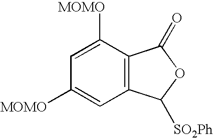 | 0/100 | Hardly soluble | Hardly soluble |
| 5 | 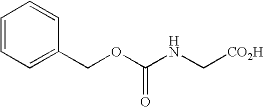 | 0/100 | 1/5 | 0/100 |
| 6 | 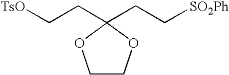 | 0/100 | 1/5 | Hardly soluble |
| 7 | 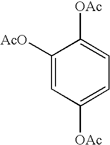 | 0/100 | 0/100 | 0/100 |
| 8 | 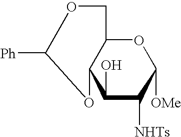 | 1/15 | 1/15 | 1/15 |
| 9 | 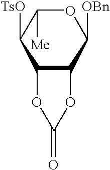 | 0/100 | 0/100 | 0/100 |
| 10 | 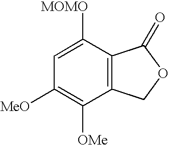 | 0/100 | 1/100 | Hardly soluble |

### Example 5

As a medium of synthesis reaction, 1,2,5-thiadiazole of the present invention was used for the synthesis of compound 10.

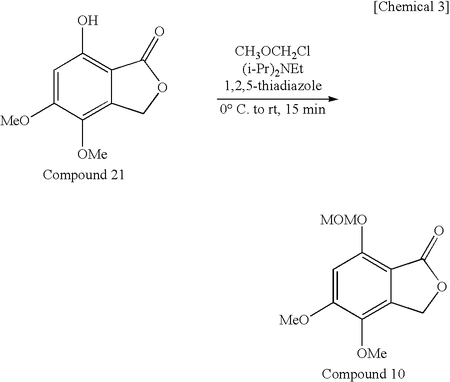

A 10.0 mg (47.6 μmol) of “compound 21” was dissolved in 200 μl of 1,2,5-thiadiazole, to which 12.4 μl (71.3 μmol) of diisopropylamine was added. 4.3 μl (57.0 μmol) of chloromethylether was added to the mixture cooled with ice, and the mixture was left at room temperature for 15 minutes. The mixture was evaporated under reduced pressure, the residue was purified by silica gel column chromatography (n-hexane:ethyl acetate=1:1), to give 12.0 mg (quantitative) of “compound 10” in the form of a while solid.

### Comparative Example 5

The same experiment as in Example 5 was performed except that the solvent was changed to dichloromethane, and compound 10 was quantitatively produced.

### Example 6

As a medium of synthesis reaction, 1,2,5-thiadiazole of the present invention was used for the synthesis of compound 23.

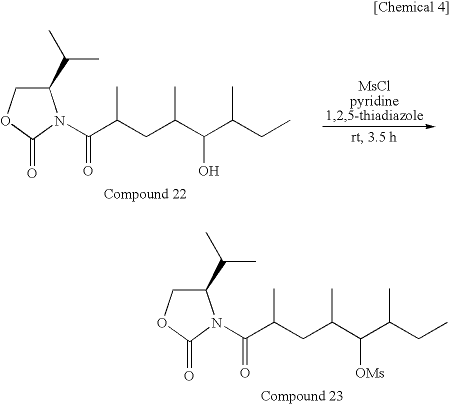

A 2.8 mg (9.0 mmol) of “compound 22” was dissolved in 150 μl of 1,2,5-thiadiazole, to which were added 7.0 μl (90 mmol) of mesyl chloride and 36 μl (0.45 mmol) of pyridine in this order, and the mixture was stirred at room temperature for 3.5 hours. Water was added to the mixture to terminate the reaction, and the mixture was further stirred for 1 hour, and concentrated under reduced pressure. 200 μl of toluene and 50 μl of water were added to the residue, the organic phase was separated, and the water phase was extracted by 50 μl of toluene. The two organic phases were combined, concentrated, and dried to give a solid. Thus, 3.7 mg (quantitative) of “compound 23” was obtained.

### Comparative Example 6

The same reaction as in Example 6 was elicited except that the solvent was changed to 1,2-dichloroethane, and compound 23 was quantitatively produced.

### Example 7

As a medium of synthesis reaction, 1,2,5-thiadiazole of the present invention was used for the synthesis of compound 25.

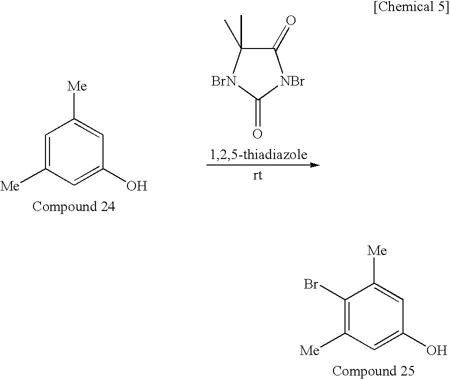

A 20.9 mg (0.171 mmol) of “compound 24” was dissolved in 210 μl of 1,2,5-thiadiazole, to which 29.3 mg (0.102 mmol) of 1,3-dibromo 5,5-dimethylhydantoin was added at room temperature under nitrogen atmosphere. On completion of reaction, 200 μl of water was added to the system, which was then subjected to separation operation. The system was evaporated under reduced pressure, and the residue was purified by silica gel column chromatography (n-hexane:ethyl acetate=10:1), to give 28.5 mg (83% yield) of “compound 25” in the form of a while solid.

### Comparative Example 7

The same reaction as in Example 7 was elicited except that the solvent was changed to dichloromethane, and compound 25 was produced at an yield of 83%.

### Example 8

As a medium of synthesis reaction, 1,2,5-thiadiazole of the present invention was used for the synthesis of compound 27.

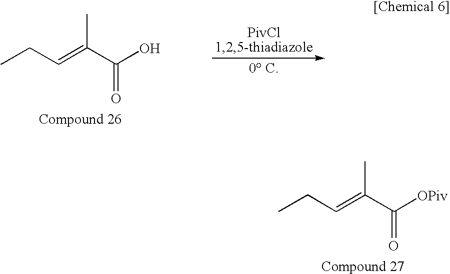

A 440 mg (3.0 mmol) of “compound 26” was dissolved in 10 ml of 1,2,5-thiadiazole, to which was added dropwise 0.81 ml (5.9 mmol) of triethylamine with stirring, followed by the gradual addition of 0.58 ml (4.7 mmol) of pivaloyl chloride. On completion of reaction, 0.6 ml of 2.0M hydrochloric acid was added to the system to terminate the reaction. 20 ml of 1,2,5-thiadiazole and 10 ml of water were added to the reaction solution, and the organic phase was separated, and the water phase was extracted by 10 ml×2 of 1,2,5-thiadiazole. The combined organic phases were dried over sodium sulfate anhydride, evaporated under reduced pressure. Thus, compound 27 was quantitatively produced as a white oil.

### Comparative Example 8

The same reaction as in Example 8 was elicited except that 1,2,5-thiadiazole was substituted for dichloromethane, and compound 27 was quantitatively produced.

### Example 9

As a medium of synthesis reaction, 1,2,5-thiadiazole of the present invention was used for the synthesis of compound 14.

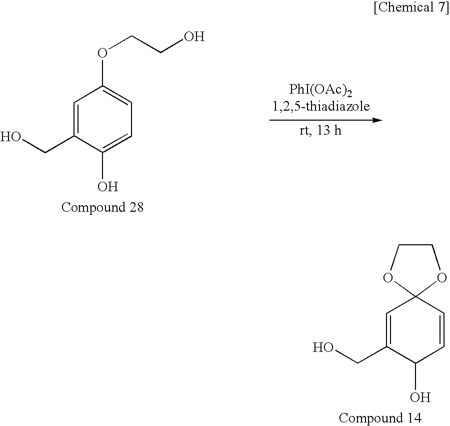

A 4.0 mg (21.7 μmol) of “compound 28” was dissolved in 1,2,5-thiadiazole, to which 7.3 mg (22.7 μmol) of PhI (OAc)₂ was added, and the mixture was stirred at room temperature for 13 hours. The mixture was added to an aqueous solution of NaHCO₃—Na₂S₂O₃, at a temperature equal to or less than 0° C., and the system, after its temperature being returned to room temperature, was extracted by 0.1 ml×2 of 1,2,5-thiadiazole. The organic phase was washed by a saturated aqueous solution of salt, and concentrated to dryness. The residue was purified by silica gel column chromatography, and thus, 2.4 mg (60% yield) of “compound 14” was obtained.

### Comparative Example 9

The same reaction as in Example 9 was elicited except that 1,2,5-thiadiazole was substituted for dichloromethane, and compound 14 was obtained at a yield of 60%.

### Example 10

As a medium of synthesis reaction, 1,2,5-thiadiazole of the present invention was used for the synthesis of compound 30.

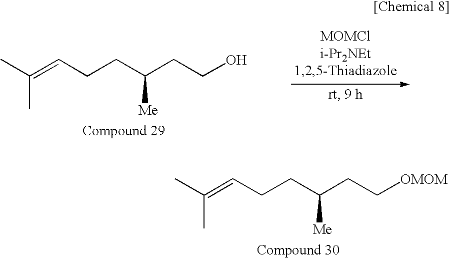

A 11.9 mg (0.0761 mmol) of “compound 29” was dissolved in 300 μl of 1,2,5-thiadiazole, to which were added 23 μl (0.128 mmol) of diisopropylethylamine and 10 μl (0.132 mmol) of methoxymethyl chloride in this order, and the mixture was allowed to stand at room temperature for 9 hours. On completion of reaction, the system was neutralized with acetic acid, and evaporated under reduced pressure to remove the solvent. The residue was purified by column chromatography (n-hexane:ethyl acetate=20:1), and thus, 4.6 mg (96% yield) of “compound 30” was obtained.

### Comparative Example 10

The same reaction as in Example 10 was elicited except that the solvent was changed to dichloromethane, and compound 30 was obtained at a yield of 93%.

### Example 11

As a medium of synthesis reaction, 1,2,5-thiadiazole of the present invention was used for the synthesis of compound 32.

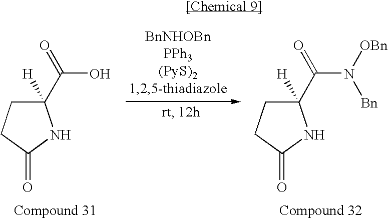

A 10.4 mg (80.5 μmol) of “compound 31” was dissolved in 1,2,5-thiadiazole, to which were added 19.5 mg (88.6 μmol) of triphenylphosphine and 23.3 mg (88.6 μmol) of dipyridyl disulfide. Thirty minutes later, 20.6 μl (96.7 μmol) of BnNHOBn was added, and the system was stirred at room temperature for 12 hours. The reaction solution was concentrated under reduced pressure, and the residue was purified by silica gel column chromatography (n-acetone: ethyl acetate=1:2), and thus, 20.8 mg (80% yield) of “compound 32” was obtained.

### Comparative Example 11

The same reaction as in Example 11 was elicited except that the solvent was changed to tetrahydrofuran, and compound 32 was obtained at a yield of 82%.

### Example 12

The washing activity of 1,2,5-thiadiazole of the present invention towards fat/oil was evaluated. A test piece consisted of a metal network having a 60 mesh density (SUS: 1 cm×3 cm) to which commercial fat/oil was uniformly applied as described below as A, B, and C.
A: vacuum pump oil (Neovac, Matsumura Oil)
B: silicon grease (Toray Grease Silicon SH111, Toray Downing Silicon)
C: silicon oil (SRX-310, Toray Downing Silicon)

The test piece was immersed in one of the fat/oil materials, removed immediately and left at room temperature for 1 hour. Then, the test piece was put in a washing bottle containing 1,2,5-thiadiazole (3 ml) of the present invention. The test piece was stirred at room temperature (20° C.) for 5 minutes, dried by exposure to hot air, and visually inspected for the presence of fat/oil residue.
A: vacuum pump oil. No residue was found.
B: silicon grease. No residue was found.
C: silicon oil. No residue was found.

### Example 13

The dissolving activity of 1,2,5-thiadiazole of the present invention towards fat/oil was evaluated using commercial fat/oil A and B as described below.
A: vacuum pump oil (Neovac, Matsumura Oil)
B: silicon grease (SRX-310, Toray Downing Silicon)

The evaluation test consisted of preparing a glass sample bottle containing 1,2,5-thiadiazole (1 ml), putting dropwise 0.1 g of one of the fat/oil materials into the bottle, and visually inspecting dissolution of the fat/oil to the solvent.
A: vacuum pump oil. Readily dissolves at room temperature (20° C.).
B: silicon grease. Readily dissolves at 50° C.

### Example 14

The washing activity of 1,2,5-thiadiazole of the present invention towards solder flux was evaluated. A test piece consisted of a metal network having a 60 mesh density (SUS: 1 cm×3 cm) to which was uniformly applied commercial solder (Sussol-F containing halogenated zinc as a main ingredient, Hakko) heated to 220° C. without being welded to the metal network. The evaluation test consisted of preparing a glass sample bottle containing 1,2,5-thiadiazole (3 ml), immersing the metal network in the solvent with stirring at room temperature (20° C.) for 3 minutes, drying the network by exposure to hot air, and visually inspecting whether any solder flux was left on the metal network.

Result

When the metal network was immersed in the solvent, solder flux readily dissolved, and three minutes later no residue was observed on the surface of the network.

### Example 15

The separating activity of 1,2,5-thiadiazole of the present invention towards an adhesive was evaluated.

A test piece was prepared by bonding a glass chip (1 cm×1 cm) to a glass plate (1 cm×3 cm) using an epoxy resin-based adhesive (High-speed Epo., Konishi) and drying the adhesive by leaving the assembly at room temperature (20° C.) for 1 hour. The separation test consisted of preparing a glass sample bottle containing 1,2,5-thiadiazole (3 ml), immersing the test piece in the solvent at room temperature (20° C.) for 60 minutes, and then evaluating the bonding activity of the adhesive.

Result

The test piece, after being immersed in the solvent of the present invention and kept there for 60 minutes, was inspected which indicated that the glass chip was separated from the glass plate, and that no residue was found either on the glass plate or on the glass chip.

### Example 16

The separating activity of 1,2,5-thiadiazole of the present invention towards photoresist was evaluated. Test pieces consisted of positive type photoresist commercially available for fine processing as described below as A and B.
A: AZ1500 (20 cp), Clariant Japan
B: OFPR-800, Tokyo Ohka Kogyo

A test piece was prepared by uniformly applying photoresist A or B on a silicon wafer to form a thin film there, radiating UV light thereto for 1 hour, and treating the wafer at 130° C. for 10 minutes to etch a wiring pattern thereon. The separation test consisted of immersing the test piece in 1,2,5-thiadiazole (3 ml) at room temperature for 60 minutes, washing the test piece with water, drying it and evaluating the separating activity of the solvent by inspecting whether any residue is left on the surface of the test piece.

Result

A: AZ1500 (20 cp). The photoresist was completely separated, and no residue was observed on the surface of the test piece.

B: OFPR-800. The photoresist was completely separated, and no residue was observed on the surface of the test piece.

### Example 17

The separating activity of 1,2,5-thiadiazole of the present invention towards paint was evaluated. A test piece consisted of a metal network having a 60 mesh density (SUS: 1 cm×3 cm) to which was uniformly applied commercial oil paint (lacquer, Kanpe Hapio), to be dried by being left at room temperature (20° C.) for 24 hours. The separation test consisted of preparing a glass sample bottle containing 1,2,5-thiadiazole (3 ml), immersing the test piece in the solvent with stirring at room temperature (20° C.) for 5 minutes, drying the test piece by exposure to hot air, and visually inspecting whether any residue is left on the surface of the test piece.

Result

No residue was observed on the surface of the metal network.

### Example 18

The dilution activity of 1,2,5-thiadiazole of the present invention towards paint was evaluated.

Evaluation of the dilution activity of 1,2,5-thiadiazole was performed by diluting 0.2 g of commercial oil paint (lacquer, Kanpe Papio) with 1,2,5-thiadiazole (0.4 ml), applying the resulting solution to a metal plate (SUS, 1 cm×3 cm), leaving the plate for 1 hour to dry, and inspecting the paint coating of the surface.

Result

The diluted solution was uniform, and maintained its uniformity even when left for 24 hours. Coating of the paint on the metal plate was uniformly achieved free from any irregularities of coloring.

What is claimed:

1. A solvent comprising at least one chosen from 1,2,5-thiadiazole compounds represented by formula (1) below:
[Chemical 1] 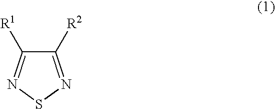
(where R¹ and R² each represent hydrogen or methyl group).

2. A solvent comprising 1,2,5-thiadiazole represented by formula (2) below:
[Chemical 2]

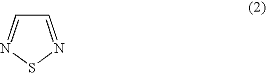

3. The solvent according to claim 1, wherein the solvent is a solvent for extraction.

4. The solvent according to claim 2, wherein the solvent is a solvent for extraction.

5. The solvent according to claim 1, wherein the solvent is a solvent serving as a medium in reaction.

6. The solvent according to claim 2, wherein the solvent is a solvent serving as a medium in reaction.

7. The solvent according to claim 1, wherein the solvent is a solvent for cleaning.

8. The solvent according to claim 2, wherein the solvent is a solvent for cleaning.

9. The solvent according to claim 1, wherein the solvent is a solvent for separation.

10. The solvent according to claim 2, wherein the solvent is a solvent for separation.

11. The solvent according to claim 1, wherein the solvent is a solvent for dilution.

12. The solvent according to claim 2, wherein the solvent is a solvent for dilution.

13. A method for extracting an organic compound using the solvent according to claim 1.

14. A method for extracting an organic compound using the solvent according to claim 2.

15. The method according to claim 13 for extracting an organic compound, wherein the method comprises contacting a solid or liquid mixture comprising the organic compound with the solvent according to claim 1 thereby extracting the organic compound, and separating a solution of the organic compound dissolved in the solvent.

16. The method according to claim 14 for extracting an organic compound, wherein the method comprises contacting a solid or liquid mixture comprising the organic compound with the solvent according to claim 2 thereby extracting the organic compound, and separating a solution of the organic compound dissolved in the solvent.

17. The method according to claim 15 for extracting an organic compound, wherein the mixture is an aqueous solution comprising the organic compound.

18. The method according to claim 16 for extracting an organic compound, wherein the mixture is an aqueous solution comprising the organic compound.

19. The method according to claim 13 for extracting an organic compound, wherein the organic compound has a polar group in its molecular structure.

20. The method according to claim 14 for extracting an organic compound, wherein the organic compound has a polar group in its molecular structure.

21. The method according to claim 15 for extracting an organic compound, wherein the organic compound has a polar group in its molecular structure.

22. The method according to claim 16 for extracting an organic compound, wherein the organic compound has a polar group in its molecular structure.

23. The method according to claim 17 for extracting an organic compound, wherein the organic compound has a polar group in its molecular structure.

24. The method according to claim 18 for extracting an organic compound, wherein the organic compound has a polar group in its molecular structure.
